(19) United States
(12) Patent Application Publication
(10) Pub. No.: US 2014/0194348 A1
(43) Pub. Date: Jul. 10, 2014

(54) METHOD OF INHIBITING PAPILLOMAVIRUS PROLIFERATION AND TREATING PAPILLOMAVIRUS INFECTION

(71) Applicant: William Bonnez, Rochester, NY (US)

(72) Inventor: William Bonnez, Rochester, NY (US)

(73) Assignee: UNIVERSITY OF ROCHESTER, Rochester, NY (US)

(21) Appl. No.: 14/148,980

(22) Filed: Jan. 7, 2014

Related U.S. Application Data
(60) Provisional application No. 61/749,673, filed on Jan. 7, 2013.

(51) Int. Cl. A61K 38/16

(52) U.S. Cl. CPC A61K 38/162
USPC 514/3.7

(57) ABSTRACT

Methods for inhibiting virus proliferation and treating an individual having a papillomavirus infection preventing viral infection are disclosed. The methods include the steps of providing a composition comprising a plurality of papillomavirus virus-like particles or capsomeres and contacting papillomavirus-infected tissue of an individual with the composition under conditions effective to inhibit papillomavirus proliferation or treat the papillomavirus infection in the individual.

The present application claims the priority benefit of U.S. Provisional Patent Application Ser. No. 61/749,673, filed Jan. 7, 2013, which is hereby incorporated by reference in its entirety.

### FIELD OF THE INVENTION

The present invention relates generally to methods of inhibiting papillomavirus proliferation and treating papillomavirus infection.

### BACKGROUND OF THE INVENTION

To initiate its life cycle, human papillomavirus (“HPV”) needs to enter the basal keratinocyte, the only dividing cell in the normal stratified epithelium, which is able to provide HPV with the necessary DNA replicative molecular machinery it lacks. Even though effective HPV vaccines are available for the prevention of papillomavirus infection, different strategies to inhibit papillomavirus proliferation and treat active papillomavirus infection still need to be addressed.

The quest for an effective method of inhibiting papillomavirus proliferation and treating papillomavirus infection has been recognized by researchers. One study by Zhang et al. reported an uncontrolled clinical trial in which patients with genital warts where sequentially assigned to one of three groups to receive intramuscular immunization with 1 (n=8), 5 (n=13), or 10 μg (n=12) of HPV-6b L1 VLPs without adjuvant (Zhang et al., “HPV6b Virus Like Particles Are Potent Immunogens Without Adjuvant in Man,” Vaccine 18(11-12):1051-1058 (2000)). There was no control group, and the patients were immunized at day 0, weeks 4 and 8. In case of treatment failure, patients were offered additional immunizations at weeks 12, 16, and 20. Complete regression of the genital warts was observed in 25 of the 33 subjects over the 20 weeks of observation. In the absence of a control group, the authors compared these results to those published in the literature, and asserted that HPV-6b L1 VLP “may accelerate [the] regression of warts.” The authors, however, were not able to establish a correlation between the clinical response and either the specific antibody or delayed type hypersensitivity response to vaccination. Further weakening their conclusion, the conjectured effect was most pronounced with the lowest dose (1 μg) of vaccine, and there were no apparent differences between the two highest dose groups (5 and 10 μg).

There is no published work describing papillomavirus VLPs as a direct therapeutic drug, in contradistinction to a vaccine that acts by the mediation of the adaptative immune response. No further studies were ever reported using HPV VLPs as a therapeutic vaccine, suggesting that the concept was not accepted. Even the senior author of the above-noted Zhang study dismissed these results eleven years later, asserting that “[a]nalysis of outcomes for subjects recruited to the major efficacy studies suggests that the currently available HPV L1 VLP vaccines are neither therapeutic for existing HPV infection nor effective in preventing progression of established HPV infection to disease” (Frazer et al., “Prevention and Treatment of Papillomavirus-Related Cancers Through Immunization,” Ann. Rev. Immunol. 29:111-38 (2011)). Supporting this assertion are the results of a study conducted in Costa-Rica and in which HPV-16 and 18 VLP immunization failed to cause the regression of existing cervical intrapithelial neoplasia (“CIN”) (Hildesheim et al., “Effect of Human Papillomavirus 16/18 L1 Viruslike Particle Vaccine Among Young Women With Preexisting Infection: A Randomized Trial,” JAMA 298(7):743-53 (2007)). Thus, there remains a need for a method of inhibiting papillomavirus proliferation and treating papillomavirus infection.

The present invention is directed to overcoming these and other deficiencies in the art using VLPs as a direct therapeutic drug.

### SUMMARY OF THE INVENTION

A first aspect of the present invention relates to a method of inhibiting papillomavirus proliferation in an individual having a papillomavirus infection. This method includes the steps of providing a composition comprising a plurality of papillomavirus virus-like particles or capsomeres; and contacting papillomavirus-infected tissue of an individual with the composition under conditions effective to inhibit papillomavirus proliferation in the individual.

According to one preferred embodiment, the papillomavirus-infected tissue is an existing lesion or tumor.

A second aspect of the present invention relates to a method of treating an individual having a papillomavirus infection. The method includes the steps of providing a composition comprising a plurality of papillomavirus virus-like particles or capsomeres; and contacting papillomavirus-infected tissue of the individual with the composition under conditions effective to treat the papillomavirus infection in the individual.

The inventor has previously established a human xenograft mouse model where the formation of HPV-induced papillomas can be inhibited if prior to HPV infection the target tissue is exposed to HPV VLPs. This preventative effect, however, did not appear to require inhibition of the viral infection itself. The present invention relies on papillomavirus VLPs or capsomeres having a direct therapeutic effect, i.e., to inhibit or cause the regression of an established papillomavirus-induced lesion or tumor. In this aspect of the invention, the Example of the present invention may utilize HPV infectious virions and VLPs that are derived either of the same genotype or a different genotype from the infecting virus. The delivery of the HPV VLPs in the present invention may be sublesional. The cutaneous HPV-infected human xenograft severe combined immunodeficiency (SCID) mouse model will be used. HPV-6 (the main causal agent of ano-genital warts) infected human foreskin grafts will be implanted on the flank of SCID mice. After a six week period, to allow the grafts to ‘take’ and grow into a condyloma, treatment will be initiated.

As an extension of this embodiment, the VLPs therapeutics encompasses any papillomavirus and papillomavirus VLPs, and more broadly, any virus and any of its homologous VLPs or capsid fragments. The VLP delivery method could be intralesional, perilesional, or systemic. The systemic delivery can be targeted either to the lesion by suitable modifications of the VLPs, or not.

Without being bound by belief, the efficacy of the present invention is based on the belief that papillomavirus VLPs when introduced in or around HPV-induced tumors or lesions will interfere with their growth, possibly inducing or permitting partial or complete regression, by a direct effect on tissue proliferation.

### BRIEF DESCRIPTION OF THE DRAWING

FIG. 1 is a schematic showing a human xenograft severe combined immunodeficiency mouse model as used for the therapeutic effect of HPV VLP on HPV-6-induced human tumors (condylomas).

### DETAILED DESCRIPTION OF THE INVENTION

The present invention is directed to methods of inhibiting papillomavirus proliferation in an individual having a papillomavirus infection and methods of treating an individual having a papillomavirus infection. The methods include the steps of providing a composition comprising a plurality of papillomavirus virus-like particles or capsomeres; and contacting papillomavirus-infected tissue of an individual with the composition under conditions effective to inhibit papillomavirus proliferation in the individual or, alternatively, to treat the individual having a papillomavirus infection.

While the methods of the present invention are directed to inhibiting papillomavirus infections, inhibition of other viruses is also contemplated. In a larger embodiment, the concept of VLP therapeutics encompasses any papillomavirus and any papillomavirus VLPs, and more broadly, any virus and any of its homologous VLPs. For example, virus infections that can be inhibited in accordance with the present invention include infections by other viruses. An exemplary non-papillomavirus infectious agent includes, but is not limited to, molluscum contagiosum virus, which is a poxvirus that causes cutaneous tumors often confused with warts.

The individual treated by the methods of the present invention can be a human or any non-human that is suffering from infection by papillomavirus or, alternatively, one of these infectious agents. Exemplary non-human individuals include, without limitation, non-human primates, horses, dairy cows and cattle, sheep, cats, dogs, rabbits, pigs, deer, and rodents (e.g., rat, mouse, and guinea pig). The individual to be treated can be female or male.

The tissue to be treated in accordance with the present invention can be any tissue that is infected by a papillomavirus (or other virus). The tissue may also contain a papillomavirus binding receptor. As used herein, the term papillomavirus binding receptor refers generically to any one or more molecular mechanisms, whether or not it is now known, that facilitate the early steps of papillomavirus infection. It is currently believed that proteoglycans, molecules made of the covalent bond of glycosaminoglycans, such as heparan sulfate, with a protein moiety, and laminin constitute the papillomavirus binding receptor.

The papillomavirus-infected tissue to be treated in accordance with the present invention may be a papillomavirus-infected lesion or tumor. In particular embodiments, the lesion or tumor may be a wart, papilloma, or pre-cancerous lesion. Examples of warts include, but are not limited to, a cutaneous wart (including a plantar wart, hand wart, and plane wart), an oral papilloma, wart or condyloma, an ano-genital wart (condyloma acuminatum), or an epidermodysplasia verruciformis lesion. Examples of papillomas include, but are not limited to, a laryngeal papilloma, a nasal papilloma, or a conjunctival papilloma. Pre-cancerous lesions that may be papillomavirus-infected tissue include, but are not limited to, a vulvar, vaginal, cervical, penile, external genitalia (including the pubis, inguinal folds, perineum, and scrotum), urethra, bladder, head, or neck area (including the mouth, the paranasal sinuses, and the larynx) lesion. In an alternative embodiment, the pre-cancerous lesion is an intraepithelial neoplasia. The pre-cancerous lesion may be, more specifically, a cervical intraepithelial neoplasia.

A variety of neoplasias are known to be associated with papillomavirus infections. For example, HPVs 3, 10, and 28 have been associated with flat warts, while HPVs 5, 8, 9, 10, 12, 14, 15, 17, 19, 20, 21, 22, 23, 24, and 25, among others, are reportedly associated with epidermodysplasia verruciformis. For a thorough classification of characterized HPV types and a discussion of genital lesions and cutaneous lesions, see de Villiers, E., “Papillomavirus and HPV Typing,” Clinics in Dermatology 15:199-206 (1997), which is hereby incorporated by reference in its entirety; see also Kremsdorf et al., “Molecular Cloning and Characterization of the Genomes of Nine Newly Recognized Human Papillomavirus Types Associated With Epidermodysplasia Verruciformis,” J. Virol. 52: 1013-1018 (1984); Beaudenon et al., “A Novel Type of Human Papillomavirus Associated With Genital Neoplasias,” Nature 321:246-249 (1986); Heilman et al., “Cloning of Human Papilloma Virus Genomic DNAs and Analysis of Homologous Polynucleotide Sequences,” J. Virol. 36:395-407 (1980); and de Villiers et al., “Molecular Cloning of Viral DNA From Human Genital Warts,” J. Virol. 40:932-935 (1981); U.S. Pat. No. 8012679 to Schlegel et al., all of which are hereby incorporated by reference in their entirety.

As indicated above, the methods of the present invention involve the use of a composition comprising a plurality of papillomavirus VLPs or capsomeres. The production of these VLPs or capsomeres via recombinant techniques is well known.

Viruses in the family Papillomaviridae are small, double-stranded, circular DNA tumor viruses. The papillomavirus virion shells consist of the L1 major capsid protein and the L2 minor capsid protein. Expression of L1 protein alone or in combination with L2 protein in eukaryotic or prokaryotic expression systems results in the assembly of VLPs, which are non-infectious and non-replicating, yet morphologically similar to natural virion. Methods for assembly and formation of human papillomavirus VLPs of the present invention are well known in the art (U.S. Pat. No. 6,153,201 to Rose et al.; U.S. Pat. No. 6,165,471 to Rose et al., WO/94/020137 to Rose et al., which are hereby incorporated by reference in their entirety). The most recent taxonomy of Papillomaviridae can be found at the NIAID Office of Cyber Infrastructure and Computational Biology, Papillomavirus Episteme (PaVE), which is hereby incorporated by reference in its entirety.

As used herein, the papillomavirus VLP or capsomere can be formed using the L1 and, optionally, L2 proteins or polypeptides from any human papillomavirus, or derivatives or fragments thereof. For a near complete listing of papillomavirus genotypes and their relatedness, see Bernard et al., “Classification of Papillomaviruses (PVs) Based on 189 PV Types and Proposal of Taxonomic Amendments,” Virol. 401:70-79 (2010), which is hereby incorporated by reference in its entirety.

The L1 protein or polypeptide can be full-length or a polypeptide fragment or derivative thereof that is competent for capsomere or VLP assembly. The L1 sequences are known for substantially all papillomavirus genotypes identified to date. The L2 sequences can also be full length or polypeptide fragments thereof. The L2 sequences are also known for substantially all papillomavirus genotypes identified to date.

The process of preparing VLPs and capsomeres basically involves the preparation of recombinant materials using known procedures, followed by the isolation and purification of these materials via known procedures.

Basically, a nucleic acid construct encoding the L1 or L1/L2 proteins or polypeptide fragments is inserted into any of the many available expression vectors and cell systems using reagents that are well known in the art. Suitable vectors include, but are not limited to, the following viral vectors such as baculovirus lambda vector system gtl 1, gt WES.tB, Charon 4, and plasmid vectors such as pBR322, pBR325, pACYC177, pACYC1084, pUC8, pUC9, pUC18, pUC19, pLG339, pR290, pKC37, pKC101, SV 40, pBluescript II SK +/− or KS +/− (see “Stratagene Cloning Systems” Catalog (1993) from Stratagene, La Jolla, Calif., which is hereby incorporated by reference in its entirety), pQE, pIH821, pGEX, pET series (see Studier et. al., “Use of T7 RNA Polymerase to Direct Expression of Cloned Genes,” Gene Expression Technology vol. 185 (1990), which is hereby incorporated by reference in its entirety), and any derivatives thereof. The DNA sequences can be cloned into the vector using standard cloning procedures known in the art, including restriction enzyme cleavage and ligation with DNA ligase as described by Sambrook et al., Molecular Cloning: A Laboratory Manual, Second Edition, Cold Spring Harbor Press, NY (2001), and Ausubel et al., Current Protocols in Molecular Biology, John Wiley & Sons, New York, N.Y. (2008), which are hereby incorporated by reference in their entirety. Recombinant molecules, including plasmids, can be introduced into cells via transformation, particularly transduction, conjugation, mobilization, or electroporation. Once these recombinant plasmids are introduced into unicellular cultures, including prokaryotic organisms and eukaryotic cells, the cells are grown in tissue culture and vectors can be replicated.

For the expression of papillomavirus L1 and L2 proteins or polypeptide fragments, and resulting capsomere and/or VLP assembly, the recombinant vectors produced above are used to infect a host cell. Any number of vector-host combinations can be employed, including yeast vectors and yeast hosts, baculovirus vectors and insect host cells, vaccinia virus vectors and mammalian host cells, or plasmid vectors in E. coli.

The capsomeres and/or VLPs of the present invention are preferably formed in Sf-9 insect cells upon expression of the L1 and optionally L2 proteins or polypeptides using recombinant baculovirus. General methods for handling and preparing baculovirus vectors and baculovirus DNA, as well as insect cell culture procedures, are outlined for example in The Molecular Biology of Baculoviruses, Doerffer et al., Eds. Springer-Verlag, Berlin, pages 31-49; Kool et al., Arch. Virol. 130: 1-16 (1993), each of which is incorporated by reference in their entirety.

Regardless of the host-vector system utilized for the recombinant expression of PV capsomeres and/or VLPs, these products can be isolated and purified using known techniques. For example, the purification of the VLPs can be achieved very simply by means of centrifugation in CsCl or sucrose gradients (Kirnbauer et al., “Papillomavirus L1 Major Capsid Protein Self-Assembles into Virus-Like Particles that are Highly Immunogenic,” Proc. Natl. Acad. Sci. (USA) 99:12180-12814 (1992); Kirnbaurer et al., “Efficient Self-Assembly of Human Papillomavirus Type 16 L1 and L1-L2 into Virus-Like Particles,” J. Virol. 67:6929-6936 (1994); Sasagawa et al., “Synthesis and Assembly of Virus-like Particles of Human Papillomaviruses Type 6 and Type 16 in Fission Yeast Schizosaccharomyces pombe,” Virology 2016:126-195 (1995); Volpers et al., “Binding and Internalization of Human Papillomavirus Type 33 Virus-Like Particles by Eukaryotic Cells,” J. Virol. 69:3258-3264 (1995); Zhou et al., “Synthesis and Assembly of Infectious Bovine Papillomavirus Particles in vitro,” J. Gen. Virol. 74:762-769 (1993); Rose et al., “Expression of Human Papillomavirus Type 11 L1 Protein in Insect Cells: in vivo and in vitro Assembly of Viruslike Particles,” J Virol. 67(4):1936-1944 (1993); Rose et al., “Serologic Differentiation of Human Papillomavirus (HPV) Types 11, 16, and 18 L1 Virus-Like Particles (VLPs),” J. Gen. Virol., 75:2445-2449 (1994), which are hereby incorporated by reference in their entirety).

Alternatively, for expression in prokaryotes such as E. coli, a GST-fusion protein or other suitable chimeric protein can be expressed recombinantly, and thereafter purified and cleaved to afford a self-assembly competent L1 polypeptide that forms capsomeres or VLPs (Chen et al., “Papillomavirus Capsid Protein Expression in Escherichia coli: Purification and Assembly of HPV11 and HPV16 L1,” J. Mol. Biol. 307:173-182 (2001), which is hereby incorporated by reference in its entirety). The resulting VLPs or capsomeres can be purified again to separate the structural assemblies from by-products.

Having purified the capsomeres and/or VLPs, these materials can be formulated into a pharmaceutically acceptable composition that can be introduced or contacted with the papillomavirus-infected tissue in accordance with the present invention. Preferably, the VLPs are introduced in an amount that is effective to inhibit papillomavirus proliferation in the individual that is to be treated in accordance with the present invention. Thus, effective amounts include an amount ranging from about 1 to about 500 μL of the VLPs or capsomeres, preferably about 5 to about 300 μL, more preferably about 10 to about 200 μL, most preferably about 15 to about 100 μL.

The method of the present aspect may include a pharmaceutically acceptable carrier and an effective amount of the papillomavirus VLPs or capsomeres.

Any number of pharmaceutically acceptable carriers can be employed depending upon the intended mode of administration. Suitable modes of administration include, any mode of that allows for delivery of the composition to a tissue comprising the HPV receptors. Exemplary modes of administration include, without limitation, administering the composition sublesionally, intralesionally, perilesionally, or systemically.

Sublesional delivery as used in accordance with the present invention refers to delivery of a compound beneath a site of papillomavirus infection (i.e., a lesion or tumor) under the skin. Intralesional delivery as used herein, refers to delivery of a compound directly to a site of papillomavirus infection (i.e., a lesion or tumor). The term includes direct injection of a compound into the lesion, and also includes delivery of a compound to a lesion on the skin where the skin is not broken during the delivery (e.g., iontophoresis delivery methods, and the like). See WO 2006/138038 to Roth et al., which is hereby incorporated by reference in its entirety.

Perilesional delivery used in the present method is delivery that is in anatomic proximity to the site of the pathologic process being treated. This type of delivery is used generally to indicate that the composition is administered in close enough anatomic proximity to allow the papillomavirus virus-like particles or capsomeres to reach the target area of pathology by local diffusion within a reasonably short period of time. See U.S. Pat. Publication No. 2003/0007972 to Tobinick, which is hereby incorporated by reference in its entirety.

Systemic administration can also be used in the present invention in conjunction with a targeted approach where the papillomavirus virus-like particles or capsomeres are modified with a targeting agent. The systemic delivery could be targeted either to the lesion by suitable modifications of the VLPs, or not. In one embodiment, the papillomavirus virus-like particle or capsomere is modified and targeted to a lesion when administered systemically. For a review of targeted systemic delivery of proteins, see Tarasov et al., “Structural Plasticity of a Transmembrane Peptide Allows Self-Assembly Into Biologically Active Nanoparticles,” PNAS 108(24):9798-803 (2011); Kaczmarczyk et al., “Protein Delivery Using Engineered Virus-Like Particles,” PNAS 108(41):16998-7003 (2011); “Virus-Like Particle Shows Promise for Cancer Drug Delivery,” News Release, University of New Mexico Cancer Center (Sep. 1, 2011); Cressey, “‘Virus-Like’ Nanoparticle Built to Target Tumours,” Nature News Blog (Aug. 19, 2012); Galaway et al., “MS2 Viruslike Particles: A Robust, Semisynthetic Targeted Drug Delivery Platform,” Mol. Pharmaceutics (e-published Nov. 14, 2012), all of which are hereby incorporated by reference in their entirety. Suitable modes of systemic administration that may be used in the present invention include, without limitation, orally, transdermally, parenterally, subcutaneously, intravenously, intramuscularly, intraperitoneally, by intranasal instillation, by intracavitary or intravesical instillation, intraocularly, intraarterially, or by application to mucous membranes.

Compositions of the present invention may be delivered in a buffer that ensures stability for the VLP and harmlessness for the tissue (e.g., one that is isotonic and at a pH close to physiologic). Examples of buffers that may be used in accordance with the present invention include, but are not limited to, sodium chloride, L-histidine, polysorbate 80, sodium borate, and water.

Solutions or suspensions can be prepared in water suitably mixed with a mild surfactant that will not disrupt the structure of the VLPs or capsomeres, such as hydroxypropylcellulose. Dispersions can also be prepared in hydroxyethylcellulose, glycerol, liquid polyethylene glycols, and mixtures thereof in oils.

Other carriers include polymeric vehicles including, without limitation, poly(ethylene-co-vinyl acetate), poly-L-lactide, poly-D-lactide, polyglycolide, poly(lactide-co-glycolide), polyanhydride, polyorthoester, polycaprolactone, polyphospagene, proteinaceous polymer, polyether, silicone or combinations thereof.

Suitable types of carriers may also include, but are not limited to, hydrogel matrices (see Kim et al., “Synthesis and Characterization of Dextran-Methacrylate and its Structure Study by SEM,” J. Biomed. Mater. Res. 49(4):517 (2000); Park et al., “Biodegradable Hydrogels for Drug Delivery,” Technomic (1993); U.S. Application Publ. No. 20060240071 to Lerner et al.; Abdool Karim et al., “Effectiveness and Safety of Tenofovir Gel, an Antiretroviral Microbicide, for the Prevention of HIV Infection in Women,” Science 329:1168-1174 (2010), all of which are hereby incorporated by reference in their entirety); buccal bioadhesive formulations, such as GelClair™ which includes water, maltodextrin, propylene glycol, polyvinylpyrrolidone, sodium hyaluronate, potassium sorbate, sodium benzoate, hydroxyethylcellulose, PEG-40 hydrogenated castor oil, disodium edetate, benzalkonium chloride, flavoring, sodium saccharin, and glycyrrhetinic acid; and liposomal delivery vehicles, a number of which are known in the art.

In one embodiment of the present invention the composition excludes separate adjuvants that would induce a powerful immune response. This is done to minimize the likelihood of inducing immune tolerance against any HPV genotypes that are utilized in commercial HPV vaccines (currently one or more of HPV-6, HPV-11, HPV-16, and HPV-18).

The compositions of the present invention may also include one or more additional therapeutic agents; buffers, neutralizing agents, agents to adjust pH; agents that preserve efficacy of the VLPs or capsomeres; coloring agents; and a balance of water or solvent. The compositions may also be used in conjunction with alternative therapeutic agents, such as, but not limited to, cryotherapy, conization, loop electrosurgical excision procedure (LEEP), electrocautery, laser vaporization or excision of papillomavirus infected cells, as well as topical treatments including podofilox (e.g., Condylox), imiquimod (e.g., Aldara or Zyclara), salicylic acid, and trichloroacetic acid.

According to one embodiment, the HPV infection to be inhibited is one which is caused by a genital-specific genotype of human papillomavirus. Exemplary genital-specific genotypes of human papillomavirus include, but are not limited to HPV-6, -11, -16, -18, -30, -31, -33, -34, -35, -39, -60, -62, -43, -64, -65, -51, -52, -53, -54, -56, -58, -59, -61, -62, -66, -67, -68, -69, -70, -71, -74, -81, -85, -86, -87, -89, -90, -91, -92, -101, -102, -103, and -106. Some of the genital-specific genotype human papillomaviruses are associated with cancer, including HPV-16, -18, -31, -33, -35, -39, -45, -51, -52, -56, -58, -59, -66, -67, -68, -73, and -82. Over 50 different HPV types have been identified in clinical lesions by viral nucleotide sequence homology studies. See, for example, Jenson et al, “Human papillomaviruses” In: Belshe, R. ed., Textbook of human virology, Second Edition, MASS: PSG, 1989:951 and Kremsdorf et al., “Molecular Cloning and Characterization of the Genomes of Nine Newly Recognized Human Papillomavirus Types Associated With Epidermodysplasia Verruciformis,” J. Virol., 52:1013-1018 (1984), which is hereby incorporated by reference in its entirety. The taxonomy of human papillomavirusess HPV1 to HPV96 is described in de Villiers, E., “Papillomavirus and HPV Typing,” Clinics in Dermatology 15:199-206 (1997) and de Villiers, E., “Classification of Papillomaviruses,” Virology 324:17-27 (2004), both of which are hereby incorporated by reference in their entirety. Numerous additional human papillomaviruses have been further characterized (see, e.g., Bernard et al., “Classification of Papillomaviruses (PVs) Based on 189 PV Types and Proposal of Taxonomic Amendments,” Virology 401:70-79 (2010), which is hereby incorporated by reference, for a listing of those papillomavirus). The HPV type determines, in part, the site of infection, the pathological features and clinical appearance as well as the clinical course of the respective lesion. U.S. Pat. No. 8,012,679 to Schlegel et al., which is hereby incorporated by reference in its entirety. VLPs and capsomeres of other HPV genotypes, whether newly discovered or previously known, or even animal papillomavirus types can also be used.

In use, the contacting of papillomavirus-infected tissue of an individual with the composition is preferably commenced as soon as possible following appearance of a tumor or lesion. In certain embodiments, the contacting may be carried out beginning several hours, days, weeks, months, or years after appearance of a lesion or tumor. In alternative embodiments, the contacting may be carried out less than or more than 72 hours after appearance of a lesion or tumor. In preferred embodiments, the contacting of the papillomavirus-infected tissue is repeated, e.g., one or more times daily, one or more times weekly, etc. over a period of weeks, months, or years, until the tumor or lesion is eradicated.

In one embodiment of the present invention, the contacting is effective to reduce the extent or size of the papillomavirus-infected tissue. The contacting may, for example, reduce the size of the papillomavirus-infected tissue by 25 percent. Alternatively, the contacting may reduce the size of the infected tissue by 50 or 75 percent. The method of the present aspect may also inhibit growth of the papillomavirus-infected tissue. In an alternative embodiment, the contacting is effective to inhibit formation of new lesions in tissue adjacent to papillomavirus-infected tissue.

In alternative embodiments, the contacting of papillomavirus-infected tissue is carried out after prior therapeutic treatment, such as surgical removal or topical treatment of infected tissue. In this embodiment, the contacting is used to inhibit further growth or spread of infection in any infected tissue that may remain following surgery.

### EXAMPLES

The Examples set forth below are for illustrative purposes only and are not intended to limit, in any way, the scope of the present invention.

### Example 1

### Materials and Methods

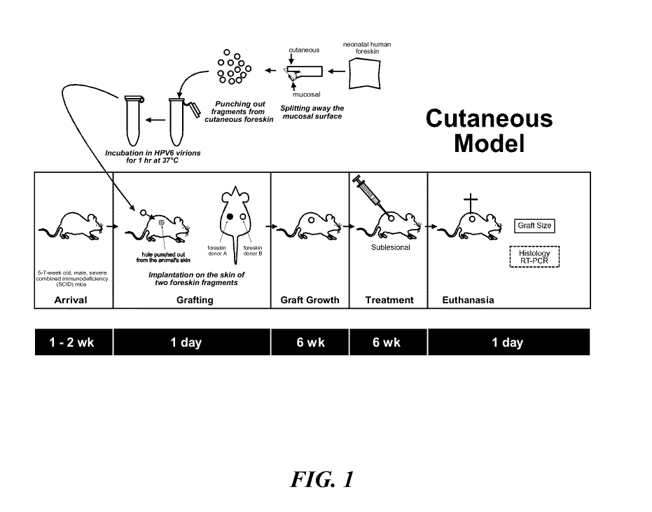

Experimental Protocol—Three different suspensions (Control (diluent buffer); HPV-6 L1 VLP; HPV-16 L1 VLP) will be evaluated in the present model of HPV-6-infected external human xenografts in the SCID mouse (FIG. 1) (Bonnez, W., “The HPV Xenograft Severe Combined Immunodeficiency Mouse Model,” Methods in Molecular Medicine 119:203-16 (2005), which is hereby incorporated by reference in its entirety). Each experiment will be done in quadruplicate.

In this animal model, the target epithelium for genital human papillomavirus infection is as in natural infections, the foreskin. The discarded foreskins will be obtained from routine neonatal circumcisions. The mucosal side of the tissue is ablated with a scalpel blade, and using a biopsy punch, discs of tissue are cut out.

These foreskin fragments are first exposed to a suspension of HPV-6 infectious virions. After challenge, they are grafted on the dorsal skin of SCID mice. Each replicate experiment uses two foreskin donors, because there is a strong foreskin donor effect that causes great variability in the ability to infect a foreskin and develop an HPV-induced papilloma. The use of two foreskin donors is to reduce this variability, while at the same time cut out enough foreskin fragments for the creation of several experimental groups. The foreskin fragments from a given donor are infected separately from those of the second donor. They are also all grafted on the same flank side of the animals, while the foreskin fragments of the other donor are implanted on the other side, one fragment per side.

Nine mice will be used in each replicate (a total of 36 mice). For each replicate, the three treatment groups each will be made of 3 mice (1 cage). Each mouse will receive on the skin of each flank one HPV-6-infected foreskin fragment. Two foreskin donors will be used, one for the fragments implanted on the left flank and a different one for the fragments implanted on the right flank. The HPV-6-infected grafts will be left to grow for 6 weeks. They will then be treated for 6 weeks with the biweekly (Mondays and Thursdays) injection of the suspension under the graft (i.e., sublesionally). Graft size will be measured before, during, and after treatment. The mice will also be weighed at the beginning of the treatment phase and every two weeks. At the end of the treatment period the mice will be sacrificed and the grafts removed, measured, and processed. They will be divided in at least two parts, one for formalin fixation, the other for freezing in liquid nitrogen and subsequent storage. The change during treatment of graft size, expressed as the composite geometric mean diameter of the two grafts borne by each animal, will be the primary endpoint. The detection of HPV-6 by histology, and HPV-6 viral expression by quantitative reverse transcriptase-polymerase chain reaction (RT-PCR) will be secondary endpoints.

Experimental Design—Six to 7 week old male FOX CHASE C.B-17 SCID (C.B-17/Icr Tac-scidfDF) will be used. The mice are purchased by the Vivarium of the University of Rochester Medical Center from Taconic (Germantown, Md.) or other vendors accredited by the American Association for Accreditation of Laboratory Animal Care (ALAAC). The experimental design is further shown in further detail in Table 1, infra, while the study schedule is shown in Table 2, infra.

TABLE 1
Experimental Design

| | | Replicate # | | | |
|---|---|---|---|---|---|
| Treatment | | 1 | 2 | 3 | 4 |
| Group 1 | Control, diluent suspension buffer | 3 mice ¹ | 3 | 3 | 3 |
| Group 2 | HPV-6 L1 VLP, 10 ng/graft/dose² | 3 | 3 | 3 | 3 |
| Group 3 | HPV-16 L1 VLP, 10 ng/graft/dose² | 3 | 3 | 3 | 3 |

¹ each mouse has two cutaneous grafts, each will be injected sub-lesionally twice a week, for 6 weeks.
²each dose will be delivered under a 20 μL volume

TABLE 2
Study Schedule

| | Week # | | | | | | | | | |
|---|---|---|---|---|---|---|---|---|---|---|
| | 0 | 2 | 4 | 6 | 7 | 8 | 9 | 10 | 11 | 12 |
| Grafting¹ | x | | | | | | | | | |
| Weighing | x | x | x | x | | x | | x | | x |
| Treatment | | | | x | x | x | x | x | x | |
| Euthanasia³ | | | | | | | | | | x |

¹Done on Monday, Tuesday, or Wednesday (the choice among three days is to allow a greater chance of having foreskins available)
²Done on Mondays and Thursdays
³Done on Mondays

HPV VLP Treatment—The test drug will be injected directly under the graft at a volume of 20 μL, using a 1 mL allergy syringe (dead volume-free) with a 27 gauge needle. The animals will be restrained but not anaesthetized for the injections. The treatment will be administered to each graft twice a week (Mondays and Thursdays) starting the sixth week after grafting and continuing for 6 weeks.

Grafting—Neonatal foreskins from routine circumcision will be collected at the nursery of a local hospital, and placed in a transport medium (Minimum Essential Medium, penicillin 25,000 U/mL, streptomycin 25 mg/mL). Two foreskin donors will be used in each experiment. Both foreskins will be processed in the same way.

In a Petri dish, the foreskin's occluded side will be removed using a scalpel, leaving the exposed skin prepared as a split-thickness graft. The foreskin will be punched out using a 4 mm biopsy punch. One fragment will be fixed in buffered formalin and will serve as control in the histologic analyses. 3-4 fragments will be snap frozen in liquid nitrogen to serve as controls in the RT-PCR assay. The other fragments will be placed into 250 μL of a suspension of HPV6_{Roch/Em-1sc/ur2b} (1:5; 2/17/11) (0.5×10⁶ DNA copies/μL), vortexed, and left to incubate for 60 minutes at 37° C. before being grafted.

For the grafting, the mouse will anesthetized in an anesthesia chamber with 5% isofluorane to 2 liters of oxygen, until no corneal or pedal reflexes could be elicited. The animal will be removed from the chamber, and the isofluoroane adjusted to 2% to 1 liter of oxygen for continued administration via nose cone for the duration of the surgical procedure.

The plane of anesthesia will be verified by the absence of a palpebral reflex, a normal respiratory rate, the absence of a corneal reflex and of a postural tone, and of withdrawal when notching the ears. The animal's back will be shaven with electric shears.

The animal ears will be notched for identification. The back skin as well as the left thigh will be prepped with a povidone iodine swab. To minimize pain after recovery, meloxicam, 2 mg/kg, will administered subcutaneously, with a 27 gauge needle, at a volume less than 100 μL.

With one hand a skin fold will be created by pinching and lifting the skin along the animal's spine and against a wood tongue depressor (Bonnez, W., “The HPV Xenograft Severe Combined Immunodeficiency Mouse Model,” Methods in Molecular Medicine 119:203-16 (2005), which is hereby incorporated by reference in its entirety). The skin fold will be punched with the other hand holding a 4 mm dermatologic disposable skin punch. This will create two 4 mm circular skin defects, one on each side of the spine. Foreskin grafts will be placed to fill these skin defects. Foreskin fragments from one donor will be grafted on one side of the animals, those from the other donor on the other side. Each graft will be maintained in place by a small Xeroform petrolatum dressing, and covered by a small, round BandAid that will be secured to the mouse skin with metal clips. The dressings will be removed two weeks later.

Animal Housing—Mice are supplied in filtered, germ-free containers, and are kept in Microisolator cages placed in a laminar-flow rack housing. There are three animals per cage. Non-recirculating fresh air is provided HEPA filtered. Temperature in the animal-housing rooms is maintained at 25.5±1° C., and the relative humidity is controlled at 68±10%. The cage contains Ultra Care Fresh bedding with sterile feed (Purina, autoclavable rodent chow) and water (pH 2.5-2.8) provided once weekly.

Rigorous techniques of sterilization and disinfection are in place. The personnel and operators are to wear a sterile gown, shoe covers, cap, mask, sterile gloves. The surgeon washes his/her hands with chlorhexidine for 5 minutes before they have to put on scrub suits, surgical gowns, caps, masks, and shoe covers.

Mice are checked daily. Each batch of SCID mice (or other immunodeficient mice) is accompanied in a separate cage by a sentinel heterozygote BALB/c mouse. Every month the sentinel mouse is bled for murine hepatitis virus serology. Every 6 mo, the sentinel mouse serum is tested for the following serologies: pneumonia virus of mice, Sendai virus, murine hepatitis virus, minute virus of mice, Theiler mouse encephalomyelitis virus, reovirus type 3, polyoma virus, ectromelia virus, K virus, mouse adenovirus, epizootic diarrhea of infant mice virus, murine cytomegalovirus, lymphochoriomeningitis virus, parvovirus, mycoplasma, pomona, and cilia-associated respiratory (CAR) bacillus. Once the last SCID mouse of the batch is sacrificed, the sentinel mouse is killed and submitted to a complete necropsy.

To minimize stress, no surgery is performed within the first 4 days after the mice arrive in the Vivarium.

After the surgery, the mouse is placed back in its cage. Free access to food and water is provided. The animal is observed until it begins to regain motor activity and grooming behavior, shows no bleeding at wound, and has a stable color and respiration. The entire procedure takes place in a room adjacent to the room where the mice are housed.

### Example 2

### Monitoring, Euthanasia, and Graft Collection

Monitoring—The animals will be monitored weekly. They will be weighed at the time of grafting, and every other week during the 12 weeks of the experiment.

Animals will be prematurely euthanatized if, during monitoring, any of the following criteria were met:
greater than 10% weight loss;
bleeding or bruising;
inability to maintain righting reflex;
inability to move about;
inability to eat or drink;
tumor greater than 30% animal's body weight.

Those animals dying unexpectedly before the date of the scheduled euthanasia will have their grafts measured and fixed in formalin, if body decomposition was not too advanced.

Methods of Euthanasia—The mice will be anesthetized by placing their cage in a ventilated chemical hood. A paper towel saturated with isofluorane will be introduced in the cage above the metal grating, and the cover will be closed. Once no corneal or pedal reflexes can be elicited the animal will be completely removed from its cage, and euthanized by cervical dislocation.

Cervical dislocation causes the cessation of respiratory movements, followed by a cardiac arrest. SCID mice are albinos, their retinal reflection is ordinarily pink. The cardiac arrest is associated with the lack of retinal vascular irrigation, causing the eye to assume a gray color. Therefore, the lack of respiration and the change in color of the mouse's eye observed over the approximately 10 minutes that the collection of grafts takes, are both an indication of the animal's death.

Graft Collection—Except for premature death or euthanasia, the animals will be sacrificed 12 weeks after graft implantation. Length, width, and height of the graft will be measured and recorded. The graft will then be removed and split in at least two parts. One part will be fixed in buffered formalin. The other part will either be left as is, or subdivided for subsequent analyses in as many fragments as size permits. These fragments will be placed in a sterile labeled vial and frozen in liquid nitrogen before being transferred to a −80° C. freezer.

### Example 3

### Histology

The formalin fixed fragments will be embedded and sectioned. One hemalum-eosin stain will be prepared. The hemalum-eosin tissue sections are reviewed for histology. The presence of at least two out of three of the following features: acanthosis, koilocytosis, or parakeratosis will be defined as evidence of HPV infection.

The tissue sections will be read by the Principal Investigator without knowing the treatment assignments. The presence of HPV as determined by histology will be a secondary endpoint.

### Example 4

### Quantitative RT-PCR

The quantitative RT-PCR and PCR will be conducted by the Functional Genomics Center of the University of Rochester. The RT-PCR will measure the magnitude of viral transcription in the samples and will be one of the secondary endpoints in the experiments, along with the histology and immunocytochemistry. The PCR will be used to quantitate the viral stock.

The following genes will be examined using Taqman chemistry with probes and primers designed to: beta-actin (GenBank X00351), HPV6 (GenBank L41216), and GAPDH (GenBank M33197).

Probes and primers (see Table 3) were designed using Primer Express v.1.0 with the following rules added to the default selection criteria provided by the software. Primers for HPV were designed to amplify a majority of viral mRNAs. First, all probes selected contain more C's than G's with no more than four consecutive bases of the same kind. Second, both forward and reverse primers are selected to have at least three of the last five bases be A's or T's preventing clamping at the 3′ primer end. The following dye combinations for probe generation are used for detection and data normalization: FAM (reporter—genes of interest), HEX (reporter—normalizer genes) and BHQ1 (non-fluorescent quencher) and ROX (reference). To determine the copy number, as a control the researcher's plasmid will be used, in pBluescript KS (Stratagene Inc., La Jolla, Calif.), containing the full genome of the HPV-6a used to infect the grafts. Following probe and primer optimization all cDNA's are diluted 1:100 with 1 μL used for each 10 μL PCR reaction containing: 5 μL of ABI 2× Universal Master Mix, 1.25 μL of each forward and reverse primers (final concentrations ranging from 200-900 nM depending on the primer set), 1 μL of probe (final concentrations ranging from 50-200 nM depending on the probe/primer set) and RNase/DNase free water per reaction. All reactions are performed in triplicate and the experiment replicated three times. Therefore, there are 9 individual reactions for each sample being tested. All reactions are run in an ABI 7900 with the following cycle parameters: 1 cycle of 50° C. (2 min) followed by 95° C. (10 min), 40 cycles of 95° C. (15 sec) followed by 60° C. (1 min). Data is collected at every temperature phase during every cycle. Raw data is analyzed using the Sequence Detection Software (ABI, Foster City Calif.).

TABLE 3
Summary of the Primers and Probes Used for the Quantitative RT-PCR and PCR Assays

| | | SEQ | Genomic |
|---|---|---|---|
| Primers/Probes | Nucleotide Sequence (5′-3′) | ID NO: | Nucleotide Position |
| HPV 6a | | | |
| Forward primer | TGCAAACGCCGCCTAGA | 1 | 3418-3434 |
| Reverse primer | CACACACAAGGCGTTGCAA | 2 | 3464-3482 |
| Probe | (FAM*)AACGAGCACGAGGAGTCCAACAGTCAC(BHQ1*) | 3 | 3436-3462 |
| GAPDH | | | |
| Forward primer | GCACCGTCAAGGCTGAGAAC | 4 | 233-252 |
| Reverse primer | ACCATCTTCCAGGAGCGAGA | 5 | 283-302 |
| Probe | (HEX*)AGCTTGTCATCAATGGAAATCCCA(BHQ1) | 6 | 257-280 |
| Beta Actin | | | |
| Forward primer | CCTGGCACCCAGCACAAT | 7 | 2717-2734 |
| Reverse primer | GCCGATCCACACGGAGTACT | 8 | 2879-2898 |
| Probe | (HEX)TCAAGATCATTGCTCCTCCTGAGCGC(BHQ1) | 9 | 2852-2877 |

*FAM and HEX are dyes, and BHQ1 is a non-fluorescent quencher used in the generation of the probes

The quantitative PCR will be used to assay the titer of the infecting viral suspension according to the method described by Wang et al. (Wang et al., “Robust Production and Passaging of Infectious HPV in Squamous Epithelium of Primary Human Keratinocytes,” Genes Develop 23(2):181-194 (2009), which is hereby incorporated by reference in its entirety). The viral suspension is digested with DNase I (Invitrogen), then inactivated by heating for 5 min at 100° C. The viral DNA is freed from the virions by digestion with proteinase K and phenol/chloroform extractions. Serial dilutions are assayed by quantitative PCR according to the method described for RT-PCR, using as a control HPV-6a plasmid.

### Example 4

### Statistical Methods

Data Management—Animal, and experimental data will be entered in a database maintained on a personal computer. Data handling and analysis will be done primarily with the statistical softwares STATISTICA version 7 (Statsoft Inc.) and Cytel Studio 9 (Cytel Corp. Cambridge, Mass.) for the statistical analysis by exact methods. The statistical power calculations will be carried out with the software PASS 6.0 for Windows (NCSS, Kaysville, Utah).

Randomization—Upon their arrival to the xenograft facility, the animal cages will receive a sequential number. This number is used to randomly assign the cages to their treatment groups. The randomization procedure will be carried out with the random number generator of the Minitab software (Minitab version 15, State College, Pa.). The randomization will be done the week before the treatment is to begin.

Analysis of Graft Size Growth (Primary Endpoint)—The primary endpoint will be graft size growth. For each mouse, an average graft size as a composite geometric mean diameter (cGMD) will be calculated:

cGMD=³√((length×width×height)_{left side}+(length×width×height)_{right side})/n where n is the number of grafts present at the beginning of treatment (week 0).

Graft size growth (GSG) will be calculated as the following percentage:

GSG=(cGMD at treatment week 6−cGMD at treatment week 0)×100/(cGMD at treatment week 0)

In case of mouse death during the second half of the treatment period, the last available cGMD will be used.

A two-way factorial design will be used. The two factors will be Treatment (3 levels) and, as a concomitant variable, Replication (treated as a fixed factor) (4 levels). The design of the sample protocol, which includes 3 treatments, 4 replicates, and 3 animals per cell, allows for an α=0.05, and a power of (1−β)=0.81, to detect an effect size of 0.55.

The primary analysis will test for the presence of a difference among the 3 treatment groups. If there is one, the analysis will then be pursued by examining pair-wise comparisons between the three different groups, using a modified Tukey Honest Significant Difference (HSD) test for unequal n's (Spjotvoll & Stoline test).

Analysis of Histology Results (Secondary Endpoint)—The results of histology, presence or absence of HPV, will be analyzed by exact logistic regression. The two independent variables will be Treatment and Foreskin Donor. The treatment variable will be treated as qualitative. Because Foreskin Donor has 8 non-ordered levels, will be made binary by the use of dummy variables. A Treatment-Foreskin Donor interaction term will not incorporated in the initial model. Should the model lack goodness-of-fit, the Treatment effect will be then analyzed by the Fisher-Freeman-Halton test without adjusting for the Foreskin Donor variable.

As with graft size, it will be asked whether HPV VLP treatment changes the histologic presence of an HPV infection.

Analysis of the quantitative RT-PCR Results (Secondary Endpoint)—The results of the quantitative RT-PCR will be analyzed by the same methods used for the graft size growth analysis. Namely the HPV RNA copy number of the two grafts implanted in a mouse will be averaged, and each experiment will be analyzed by a 2-way ANOVA, the two factors being Treatment and Replicate (to control for the foreskin donor effect), each treated as fixed.

In all statistical analyses two-sided p values equal or less than 0.05 will be considered significant.

Analysis of Animal Survival and Weight Change—As part of the evaluation of drug toxicity, any differences in the survival of the animals during the treatment period will be examined by the Kruskal-Wallis test. The animals' percentage weight gains during the treatment phase will be analyzed and compared among groups by one-way ANOVA. If an effect is observed, pair-wise differences between groups will be looked for.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of inhibiting papillomavirus proliferation in an individual having a papillomavirus infection, the method comprising:
providing a composition comprising a plurality of papillomavirus virus-like particles or capsomeres; and
contacting papillomavirus-infected tissue of an individual with the composition under conditions effective to inhibit papillomavirus proliferation in the individual.

2. The method according to claim 1, wherein the papillomavirus-infected tissue is a lesion or tumor.

3. The method according to claim 2, wherein the lesion is a wart, papilloma, or pre-cancerous lesion.

4. The method according to claim 3, wherein the wart is a cutaneous wart, head and neck area papilloma, wart or condyloma, ano-genital wart, or an epidermodysplasia verruciformis lesion.

5. The method according to claim 3, wherein the pre-cancerous lesion is a vulvar, vaginal, anal, cervical, penile, external genitalia, urethra, bladder, or head and neck area lesion.

6. The method according to claim 1, wherein the papillomavirus virus-like particles or capsomeres comprise an L1 polypeptide and optionally an L2 polypeptide.

7. The method according to claim 1, wherein the contacted papillomavirus-infected tissue is an ano-genital tissue or an oral tissue.

8. The method according to claim 1, wherein said contacting is carried out by administering the composition sublesionally, intralesionally, perilesionally, or systemically.

9. The method according to claim 1, wherein said contacting is effective to reduce the extent or size of papillomavirus-infected tissue.

10. The method according to claim 1, wherein said contacting is effective to inhibit growth of the papillomavirus-infected tissue.

11. The method according to claim 1, wherein said contacting is effective to inhibit formation of new lesions in tissue adjacent to papillomavirus-infected tissue.

12. A method of treating an individual having a papillomavirus infection, the method comprising:
providing a composition comprising a plurality of papillomavirus virus-like particles or capsomeres; and
contacting papillomavirus-infected tissue of the individual with the composition under conditions effective to treat the papillomavirus infection in the individual.

13. The method according to claim 12, wherein the papillomavirus-infected tissue is a lesion or tumor.

14. The method according to claim 13, wherein the lesion is a wart, papilloma, or pre-cancerous lesion.

15. The method according to claim 14, wherein the wart is a cutaneous wart, head and neck area papilloma, wart or condyloma, ano-genital wart, or an epidermodysplasia verruciformis lesion.

16. The method according to claim 14, wherein the pre-cancerous lesion is a vulvar, vaginal, anal, cervical, penile, external genitalia, urethra, bladder, or head and neck area lesion.

17. The method according to claim 12, wherein the papillomavirus virus-like particles or capsomeres comprise an L1 polypeptide and optionally an L2 polypeptide.

18. The method according to claim 12, wherein the contacted papillomavirus-infected tissue is an ano-genital tissue or an oral tissue.

19. The method according to claim 12, wherein said contacting is carried out by administering the composition sublesionally, intralesionally, perilesionally, or systemically.

20. The method according to claim 12, wherein said contacting is effective to reduce the extent or size of papillomavirus-infected tissue.

21. The method according to claim 12, wherein said contacting is effective to inhibit growth of the papillomavirus-infected tissue.

22. The method according to claim 12, wherein said contacting is effective to inhibit formation of new lesions in tissue adjacent to papillomavirus-infected tissue.


SEQUENCE LISTING (9 sequences)

SEQ ID NO: 1 - DNA, 17 bp, artificial
  HPV 6a forward primer
tgcaaacgccgcctaga

SEQ ID NO: 2 - DNA, 19 bp, artificial
  HPV 6a reverse primer
cacacacaaggcgttgcaa

SEQ ID NO: 3 - DNA, 27 bp, artificial
  HPV 6a probe
aacgagcacgaggagtccaacagtcac

SEQ ID NO: 4 - DNA, 20 bp, artificial
  GAPDH forward primer
gcaccgtcaaggctgagaac

SEQ ID NO: 5 - DNA, 20 bp, artificial
  GAPDH reverse primer
accatcttccaggagcgaga

SEQ ID NO: 6 - DNA, 24 bp, artificial
  GAPDH probe
agcttgtcatcaatggaaatccca

SEQ ID NO: 7 - DNA, 18 bp, artificial
  beta actin forward primer
cctggcacccagcacaat

SEQ ID NO: 8 - DNA, 20 bp, artificial
  beta actin reverse primer
gccgatccacacggagtact

SEQ ID NO: 9 - DNA, 26 bp, artificial
  beta actin probe
tcaagatcattgctcctcctgagcgc